(19) United States
(12) Patent Application Publication
(10) Pub. No.: US 2013/0006215 A1
(43) Pub. Date: Jan. 3, 2013

(54) FLUID DISPENSING DEVICE

(75) Inventors: Todd William Sharratt, Stillwater, MN (US); Troy Allen Pongratz, Minneapolis, MN (US)

(73) Assignee: NORDSON CORPORATION, Westlake, OH (US)

(21) Appl. No.: 13/579,117

(22) PCT Filed: Mar. 24, 2011
(86) PCT No.: PCT/US11/29743

§ 371 Date: Aug. 15, 2012

Related U.S. Application Data
(60) Provisional application No. 61/317,193, filed on Mar. 24, 2010.

(51) Int. Cl. A61M 5/315

(52) U.S. Cl. 604/500; 604/228; 604/222

(57) ABSTRACT

A fluid dispensing device (10) for delivering a dose of fluid into a surgical site includes a tube (12) with a lumen (22) for receiving the dose of fluid, a housing (14) coupled to the tube, and a flexible plunger (24) at least partially disposed in the housing (14). The fluid dispensing device (10) also includes an actuation device (66, 140) engaging the flexible plunger (24) and a trigger (26) operable to cause the actuation device (66, 140) to move the flexible plunger (24). The actuation device (66, 140) may be toggled between a first operational mode in which movement of the trigger (26) advances the flexible plunger (24) into the lumen (22) and a second operational mode in which movement of the trigger (26) retracts the flexible plunger (24) from the lumen (22).

### CROSS-REFERENCE TO RELATED APPLICATION

This application claims the priority of U.S. Provisional Patent Application Ser. No. 61/317,193, filed on Mar. 24, 2010 (pending), the disclosure of which is incorporated by reference herein.

### TECHNICAL FIELD

This invention generally relates to fluid dispensing devices, and particularly to a fluid dispensing device configured to deliver fluids to a surgical site.

### BACKGROUND

In the medical field, a surgeon routinely needs to deliver a drug or another fluid to a surgical site in a patient. Conventional syringes that are manually or non-manually actuated are often used to deliver these fluids to the surgical site. However, many surgeries are now performed using minimally-invasive techniques such as laparoscopic procedures and small incisions to reduce impact on the patient and corresponding recovery time. In these minimally-invasive procedures, fluid delivery via conventional syringes is not a viable option because the syringe is too large to access the surgical site through the small incision in the patient. Consequently, alternative methods of delivering fluids to the surgical site must be employed in these circumstances.

In less invasive procedures, fluid is generally delivered to the surgical site with an elongate small-diameter tube having sufficient length to contain enough fluid for the surgical site. Fluid in the tube is dispensed with a rigid elongate plunger extending proximally from the tube. However, the elongate tube and the rigid elongate plunger collectively define a significant length so that an entire dose of the fluid may be contained in a single elongate tube. To this end, the combined tube and plunger may be difficult or unwieldy to work with in an operating room or other space-constrained surgical setting.

There is a need, therefore, for a fluid dispensing device that addresses these and other problems associated with conventional fluid dispensing devices.

### SUMMARY

In one embodiment of the invention, a fluid dispensing device is configured to deliver a dose of fluid into a surgical site. The device includes a tube having a proximal end, a distal end, and a lumen extending from the proximal end to the distal end. The lumen receives the dose of fluid. The device also includes a housing coupled to the tube at the proximal end and a flexible plunger disposed at least partially in the housing. The device also includes an actuation device coupled with the housing and engaging the flexible plunger. A trigger is coupled for movement relative to the housing and is operable to cause the actuation device to move a distal portion of the flexible plunger into the lumen to force the dose of fluid out of the distal end of the tube.

The actuation device may include first and second operational modes. In the first operational mode, movement of the trigger causes advancement of the flexible plunger into the lumen. In the second operational mode, movement of the trigger causes retraction of the flexible plunger from the lumen. The flexible plunger may further include a proximal portion that is curved or coiled when disposed within the housing, such that the overall length of the fluid dispensing device is shortened. The device may also include a Y-shaped receptacle coupling the proximal end of the tube to the housing, the receptacle including an auxiliary inlet port for directed a new dose of fluid into the lumen when the flexible plunger is retracted into the housing.

In another embodiment of the invention, a fluid dispensing device again includes a tube having a proximal end, a distal end, and a lumen extending from the proximal end to the distal end. The device also includes a housing coupled to the tube at the proximal end, a plunger at least partially disposed in the housing, and an actuation device coupled with the housing and engaging the plunger. The fluid dispensing device also includes a trigger coupled for movement relative to the housing and operable to cause the actuation device to move a distal portion of the plunger. In a first operational mode of the actuation device, movement of the trigger causes advancement of the plunger into the lumen. In a second operational mode of the actuation device, movement of the trigger causes retraction of the plunger from the lumen.

The actuation device may also include first and second actuating members operatively coupled to the trigger. The first actuating member is configured to transfer movement of the trigger to advance the plunger in the first operational mode, while the second actuating member is configured to transfer movement of the trigger to retract the plunger in the second operational mode. For example, the first and second actuating members may include first and second one-way bearings selectively coupling transfer gears to a drive shaft in the actuation device. In another example, the first and second actuating members include first and second gripping collars selectively engaging the plunger.

In yet another embodiment of the invention, a method of dispensing a dose of fluid into a surgical site with a fluid dispensing device is provided. The method includes placing an actuation device of the fluid dispensing device in a first operational mode in which movement of a trigger causes advancement of a flexible plunger into a lumen containing the dose of fluid. The method also includes actuating the trigger to move the actuation device and moving the flexible plunger with the actuation device from a curved configuration within a housing to a straightened configuration within the lumen to force the dose of fluid from the lumen. The method may also include placing the actuation device in a second operational mode in which movement of the trigger causes retraction of the flexible plunger from the lumen. Thus, the lumen may be reloaded with a new dose of fluid when the flexible plunger is retracted from the lumen back to a curved configuration.

### BRIEF DESCRIPTION OF THE DRAWINGS

FIG. 1 is a perspective view of one embodiment of a fluid dispensing device.

FIG. 2 is a perspective view of the fluid dispensing device of FIG. 1, with the housing shown in phantom to reveal interior drive components.

FIG. 3 is a perspective view of the interior drive components of the fluid dispensing device of FIG. 1, with several components shown in phantom.

FIG. 4A is a top view of the interior drive components of the fluid dispensing device of FIG. 1 in a first operational mode.

FIG. 4B is a detailed cross-sectional view of the transfer gears of the fluid dispensing device of FIG. 4A, with the drive shaft in a first position.

FIG. 4C is a side cross-sectional view of the fluid dispensing device of FIG. 4A along line 4C-4C in FIG. 2, with the housing shown in phantom and the trigger in a first position.

FIG. 4D is a side of the fluid dispensing device of FIG. 4A, with the housing shown in phantom and the trigger in a second position.

FIG. 5A is a top view of the interior drive components of the fluid dispensing device of FIG. 1 in a second operational mode.

FIG. 5B is a detailed cross-sectional view of the transfer gears of the fluid dispensing device of FIG. 5A, with the drive shaft in a second position.

FIG. 5C is a side cross-sectional view of the fluid dispensing device of FIG. 5A along line 5C-5C in FIG. 2, with the housing shown in phantom and the trigger in a first position.

FIG. 5D is a side of the fluid dispensing device of FIG. 5A, with the housing shown in phantom and the trigger in a second position.

FIG. 6 is a perspective view of another embodiment of a fluid dispensing device.

FIG. 7A is a side cross-sectional view of the fluid dispensing device of FIG. 6 along line 7A-7A, with the actuation device in a first operational mode and the trigger in a first position.

FIG. 7B is a detailed side cross-sectional view of the actuation device as shown in FIG. 7A.

FIG. 8A is a perspective view of the actuation device of the fluid dispensing device of FIG. 6.

FIG. 8B is a perspective exploded view of the actuation device of the fluid dispensing device of FIG. 6.

FIG. 9A is a side cross-sectional view of the fluid dispensing device of FIG. 6, with the actuation device in a first operational mode and the trigger in a second position to advance the plunger.

FIG. 9B is a detailed side cross-sectional view of the actuation device as shown in FIG. 9A.

FIG. 10A is a side cross-sectional view of the fluid dispensing device of FIG. 6, with the actuation device in a second operational mode and the trigger in a first position.

FIG. 10B is a detailed side cross-sectional view of the actuation device as shown in FIG. 10A.

FIG. 11A is a side cross-sectional view of the fluid dispensing device of FIG. 6, with the actuation device in a second operational mode and the trigger in a second position.

FIG. 11B is a detailed side cross-sectional view of the actuation device as shown in FIG. 11A.

FIG. 12A is a side cross-sectional view of the fluid dispensing device of FIG. 6, with the actuation device in a second operational mode and the trigger in a third position to retract the plunger.

FIG. 12B is a detailed side cross-sectional view of the actuation device as shown in FIG. 12A.

### DETAILED DESCRIPTION

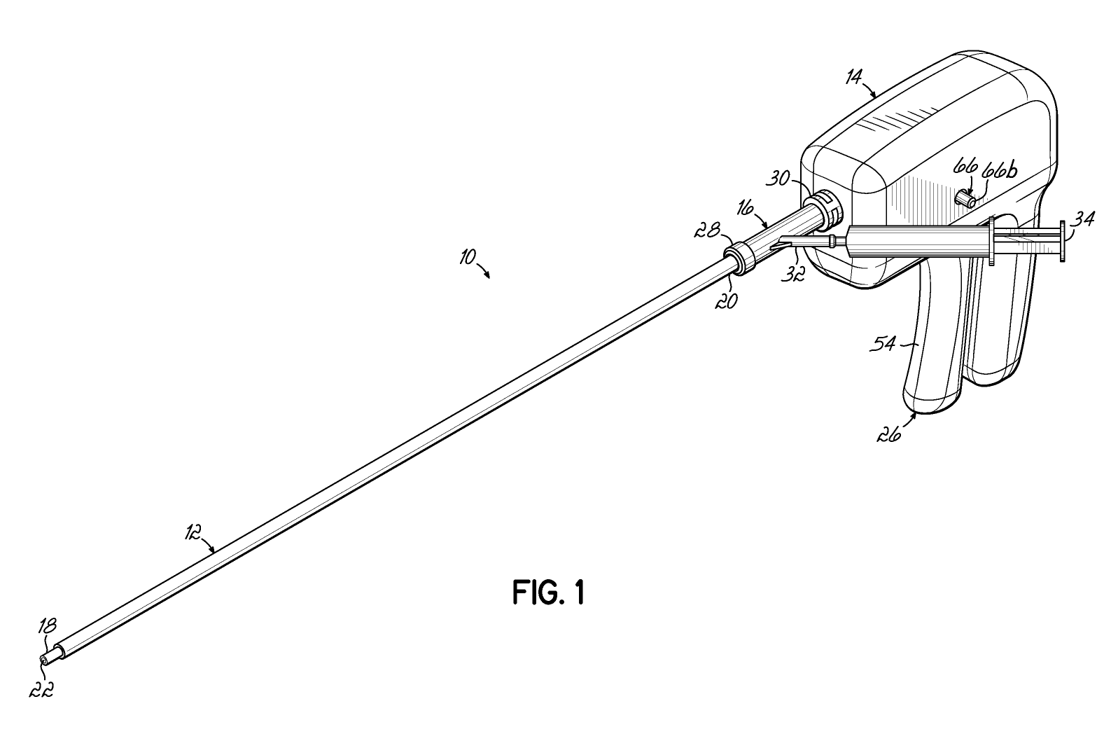

FIGS. 1-5D illustrate one embodiment of a fluid dispensing device 10 configured to deliver a dose of fluid into a surgical site. More particularly, the fluid dispensing device 10 is operable to dispense the dose of fluid into small incision surgical sites such as those used in minimally-invasive surgery and laparoscopic procedures. As shown in FIG. 1, the fluid dispensing device 10 includes an elongate tube 12 for holding the dose of fluid, a housing 14, and a Y-shaped receptacle 16 coupling the elongate tube 12 to the housing 14. The elongate tube 12 is generally rigid and includes a distal end 18, a proximal end 20 coupled to the Y-shaped receptacle 16, and a lumen 22 extending from the distal end to the proximal end 20. The housing 14 is configured to receive a plunger 24 (not shown in FIG. 1) that may be advanced into the lumen 22 to force the dose of fluid from the distal end 18 of the tube 12. The plunger 24 may also be retracted from the lumen 22 to permit a new dose of fluid to be delivered into the lumen 22. The fluid dispensing device 10 also includes a trigger 26 coupled to the housing 14 and operable to advance or retract the plunger 24 as described in further detail below.

Also shown in FIG. 1, the Y-shaped receptacle 16 includes a first end 28 configured to receive the proximal end 20 of the tube 12, a second end 30 configured to be received in the housing 14, and an auxiliary port 32 between the first and second ends 28, 30. The auxiliary port 32 is in fluid communication with the lumen 22 when the tube 12 is coupled to the Y-shaped receptacle 16. As such, the auxiliary port 32 may be coupled to a syringe 34 or other known medicament container to reload the elongate tube 12 with fluid when the plunger 24 is retracted. It will be understood that the elongate tube 12 may be directly coupled to the housing 14 without the Y-shaped receptacle 16 in other non-illustrated embodiments.

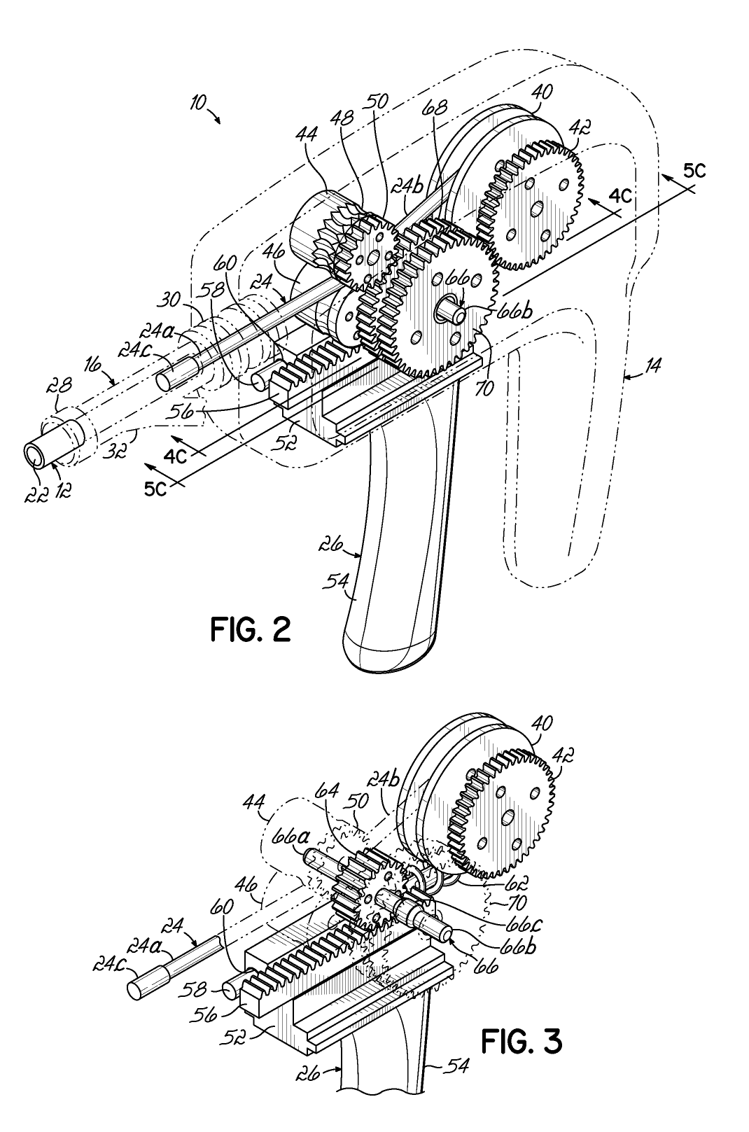

The interior drive components of the fluid dispensing device 10 are shown more clearly in FIGS. 2 and 3, where the tube 12, the housing 14, and the receptacle 16 are shown in phantom. To this end, FIG. 2 illustrates that the plunger 24 includes a distal portion 24a and a proximal portion 24b, at least the proximal portion 24b being flexible so that the plunger 24 may be coiled around a reel 40 mounted within the housing 14. The reel 40 includes a reel gear 42 configured to be driven to retract the plunger 24 as described in further detail below. The plunger 24 also extends between a gripper wheel 44 and an idler wheel 46. The gripper wheel 44 is mounted within the housing 14 and includes gripping teeth 48 for engaging the plunger 24 and a gripper wheel gear 50 configured to be driven to advance the plunger 24 as described in further detail below. To this end, the reel 40 and the gripper wheel 44 collectively define at least a portion of an actuation device for moving the plunger 24 into and out of the lumen 22 of the tube 12. Although the actuation device components are described as being coupled to or mounted in the housing 14, it will be appreciated that these couplings or mountings may be direct or indirect as well understood in the art.

The trigger 26 further includes a body portion 52 mounted for sliding movement within the housing 14, a handle portion 54 extending outside the housing 14, and a rack gear 56 disposed on the body portion 52. In the illustrated embodiment, the housing 14 includes an elongate rod 58 and the body portion 52 of the trigger 26 includes an aperture 60 configured to slide along the elongate rod 58. The elongate rod 58 may also carry a compression spring 62 configured to bias the trigger 26 against the housing 14 towards the position shown in FIG. 2. As the trigger 26 slides with respect to the housing 14, the rack gear 56 engages and rotates a drive gear 64 mounted within the housing 14 as shown in FIG. 3. The drive gear 64 selectively actuates the reel 40 and the gripper wheel 44 to move the plunger 24.

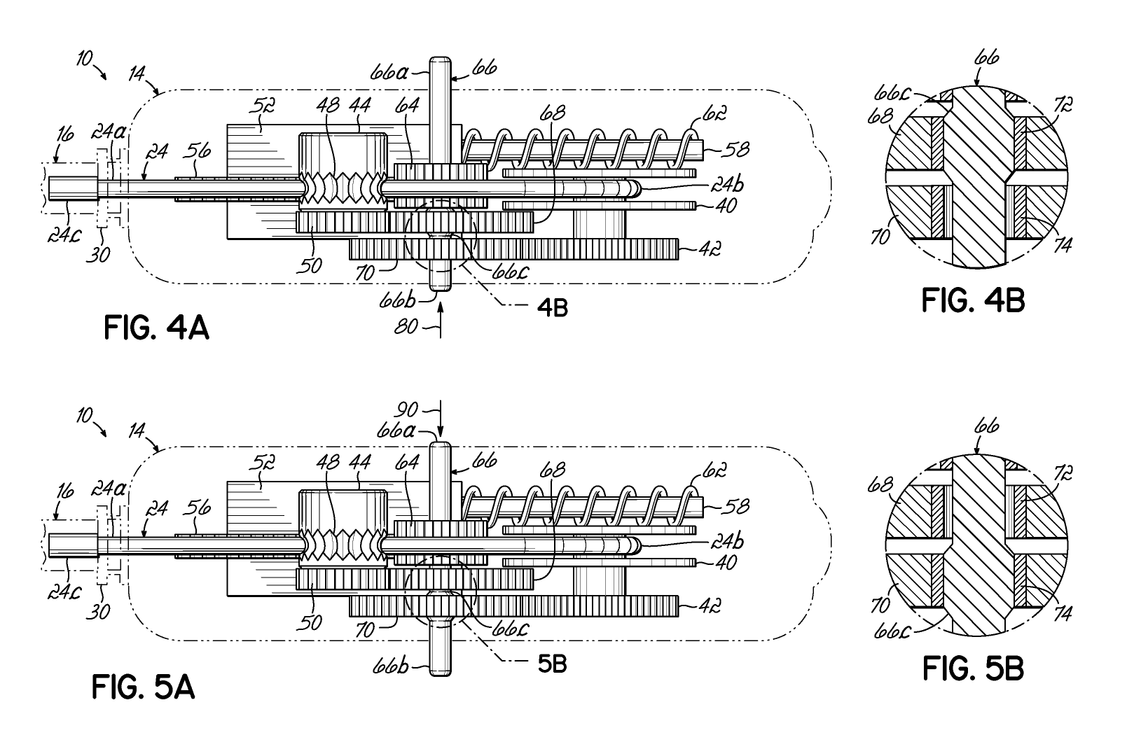

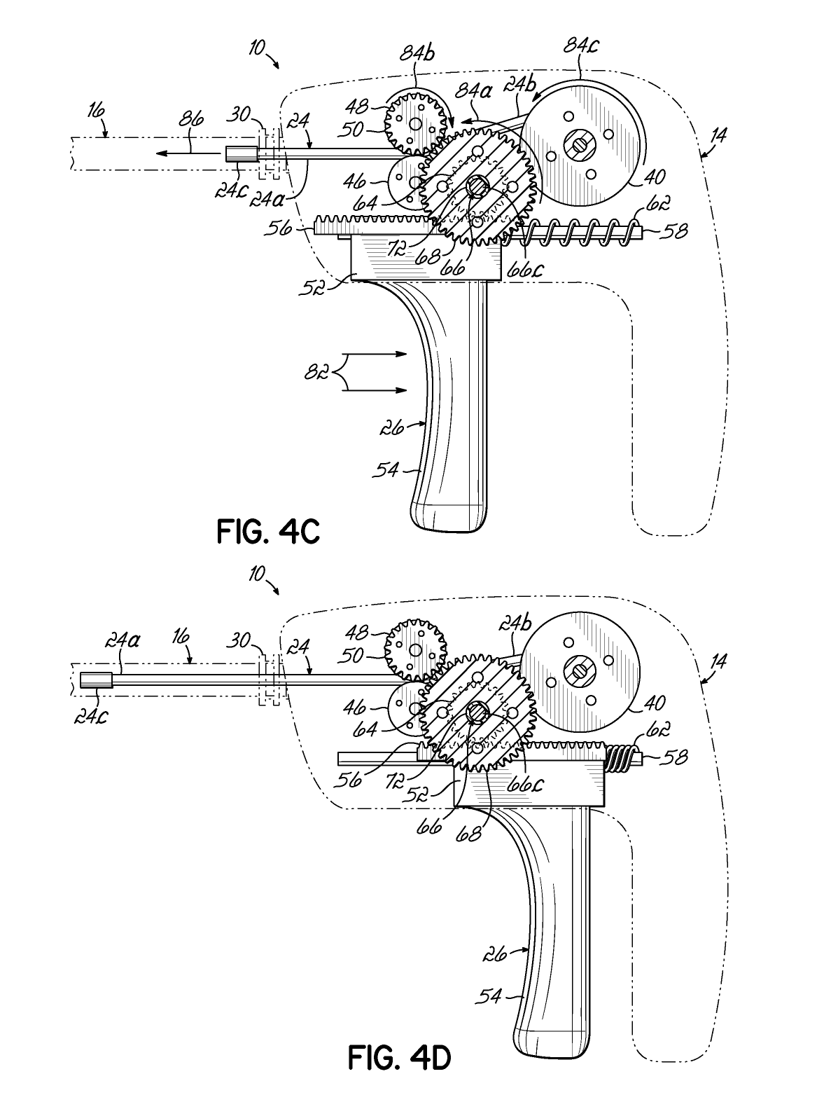

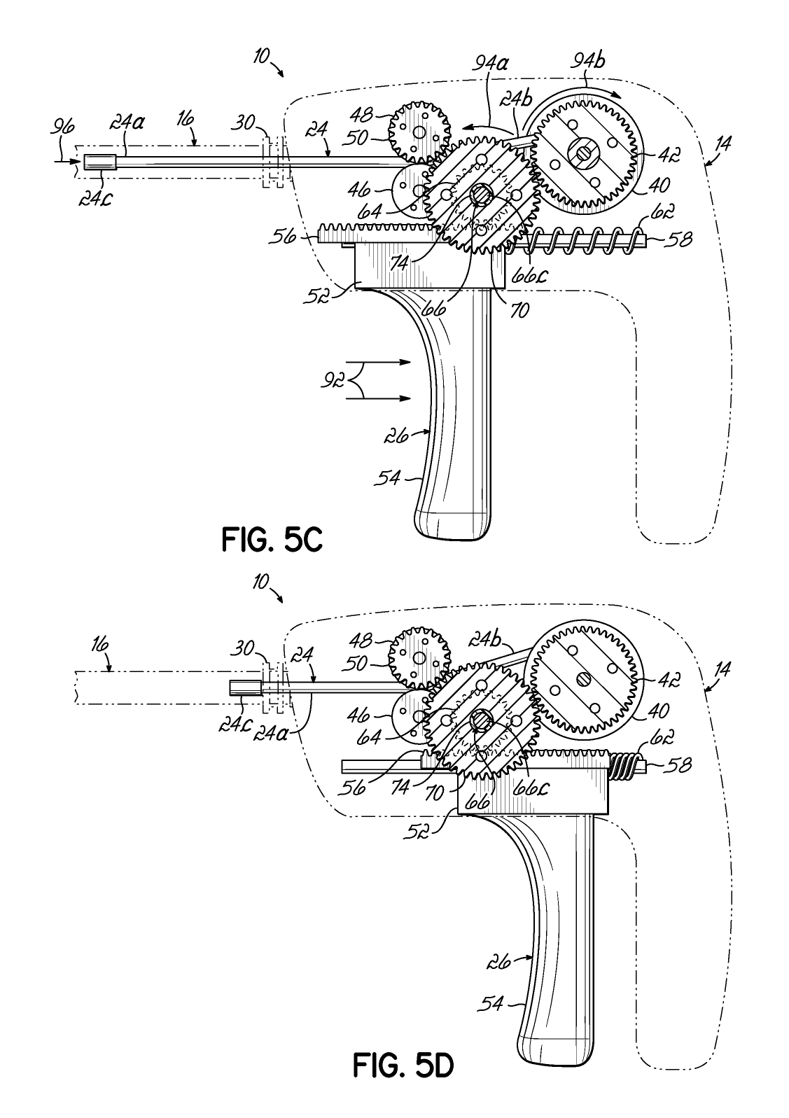

As shown in FIGS. 2 and 3, the drive gear 64 is mounted on a drive shaft 66 configured to rotate with the drive gear 64. The drive shaft 66 includes a first end 66a and a second end 66b each extending outside the housing 14. The drive shaft 66 is configured to be slid transversely in the housing 14 to toggle the actuation device between a first operational mode (FIGS. 4A-4D) and a second operational mode (FIGS. 5A-5D), each of which is described in further detail below. The drive shaft 66 also includes an output portion 66c having a larger diameter than the remainder of the drive shaft 66. The output portion 66c of the drive shaft 66 is configured to selectively engage one of a first transfer gear 68 and a second transfer gear 70 disposed within the housing 14. In this regard, the output portion 66c of the drive shaft 66 engages the first transfer gear 68 in the first operational mode and engages the second transfer gear 70 in the second operational mode. Thus, only one of the first and second transfer gears 68, 70 is actively driven by movement of the trigger 26 at any instance in time.

The first transfer gear 68 is engaged with the gripper wheel gear 50 and is operable to drive the gripper wheel 44. Therefore, when the output portion 66c of the drive shaft 66 is engaged with the first transfer wheel 68, movement of the trigger 26 will actuate rotation of the gripper wheel 44. The second transfer gear 70 is engaged with the reel gear 42 and is operable to drive the reel 40. Therefore, when the output portion 66c of the drive shaft 66 is engaged with the second transfer wheel 70, movement of the trigger 26 will actuate rotation of the reel 40. The actuation device/mechanism of the fluid dispensing device 10 may be readily toggled between these two operational modes by pushing on the first end 66a or the second end 66b of the drive shaft 66. As such, the trigger 26 may be moved to actuate both advancement and retraction of the plunger 24.

The first transfer gear 68 further includes a first one-way bearing 72 configured to directly engage the output portion 66c of the drive shaft 66 in the first operational mode. The first one-way bearing 72 permits the drive shaft 66 to rotate the first transfer gear 68 in only one direction as understood in the bearing field. The first one-way bearing 72 is configured to only permit driven rotation of the first transfer gear 68 in such a manner that drives the gripper wheel gear 50 and gripper wheel 44 to advance the plunger 24 into the lumen 22. Similarly, the second transfer gear 70 further includes a second one-way bearing 74 configured to directly engage the output portion 66c of the drive shaft 66 in the second operational mode. The second one-way bearing 74 is oriented in the same direction as the first one-way bearing 72. In this regard, the second one-way bearing 74 permits the drive shaft 66 to rotate the second transfer gear 70 in only one direction. Thus, the second one-way bearing 74 is configured to only permit driven rotation of the second transfer gear 70 in such a manner that drives the reel gear 42 and reel 40 to retract the plunger 24 from the lumen 22 and wrap the plunger 24 in a curved or coiled condition on the reel 40.

The operation of the fluid dispensing device 10 in the first operational mode is further illustrated in FIGS. 4A-4D. To place the fluid dispensing device 10 in the first operational mode, the second end 66b of the drive shaft 66 may be pushed from outside the housing 14 as indicated by arrow 80 in FIG. 4A. In this position, the output portion 66c of the drive shaft 66 engages the first transfer wheel 68 at the first one-way bearing 72 as shown most clearly in FIG. 4B. Consequently, the second transfer wheel 70 is not engaged with the drive shaft 66 and is free to rotate independently. As described briefly above, the fluid dispensing device 10 is configured to advance the plunger 24 in this first operational mode.

As shown in FIG. 4C, the trigger 26 begins in a forward position and the plunger 24 begins in a first position nearly retracted completely from the tube 12. When a user applies manual force to move the trigger 26 rearwardly as shown by arrows 82, the rack gear 56 forces the drive gear 64 and drive shaft 66 to rotate the first transfer gear 68 in a counterclockwise direction (arrow 84a) as viewed in FIG. 4C. The first one-way bearing 72 permits this driven rotation in the counterclockwise direction such that the first transfer gear 68 causes clockwise rotation (arrow 84b) of the gripper wheel gear 50 and gripper wheel 44. The gripping teeth 48 grasp the plunger 24 and force the plunger 24 to advance as shown by arrow 86 with this clockwise rotation. It will be appreciated that the advancement of the plunger 24 automatically forces counterclockwise rotation (arrow 84c) of the reel 40, which is also enabled by the free rotation of the second transfer gear 70. If the trigger 26 is fully moved to the rearward position shown in FIG. 4D, the plunger 24 is advanced into the lumen 22 of the tube 12 as shown in FIG. 4D.

From the position shown in FIG. 4D, the compression spring 62 will bias the trigger 26 to slide back to the position shown in FIG. 4C. While the trigger 26 moves forward, the drive shaft 66 rotates in an opposite direction but the first one-way bearing 72 prevents corresponding rotation of the first transfer gear 68. To this end, the trigger 26 can return to the position shown in FIG. 4C without causing retraction of the plunger 24. The process may then be repeated until the plunger 24 is fully advanced into the tube 12 to dispense the dose of fluid. It will also be appreciated from FIGS. 4C and 4D that the first transfer gear 68 is sized larger than the gripper wheel gear 50 such that a step-up type of transmission is formed, transferring one unit of movement of the trigger 26 into two or more units of movement for the plunger 24. The gear ratios of the actuation device/mechanism may be modified without departing from the scope of this invention.

The operation of the fluid dispensing device 10 in the second operational mode is further illustrated in FIGS. 5A-5D. To place the fluid dispensing device 10 in the second operational mode, the first end 66a of the drive shaft 66 may be pushed from outside the housing 14 as indicated by arrow 90 in FIG. 5A. In this position, the output portion 66c of the drive shaft 66 engages the second transfer wheel 70 at the second one-way bearing 74 as shown most clearly in FIG. 5B. Consequently, the first transfer wheel 68 is not engaged with the drive shaft 66 and is free to rotate independently. As described briefly above, the fluid dispensing device 10 is configured to retract the plunger 24 in this second operational mode.

As shown in FIG. 5C, the trigger 26 begins in a forward position and the plunger 24 begins in a first position advanced into the lumen 22 of the tube 12. When a user applies manual force to move the trigger 26 rearwardly as shown by arrows 92, the rack gear 56 forces the drive gear 64 and drive shaft 66 to rotate the second transfer gear 70 in a counterclockwise direction (arrow 94a) as viewed in FIG. 5C. The second one-way bearing 74 permits this driven rotation in the counterclockwise direction such that the second transfer gear 70 causes clockwise rotation (arrow 94b) of the reel gear 42 and the reel 40. The reel 40 coils the plunger 24 and forces the plunger 24 to retract as shown by arrow 96 with this clockwise rotation. If the trigger 26 is fully moved to the rearward position shown in FIG. 5D, the plunger 24 is retracted from the lumen 22 of the tube 12 as shown in FIG. 5D.

From the position shown in FIG. 5D, the compression spring 62 will bias the trigger 26 to slide back to the position shown in FIG. 5C. While the trigger 26 moves forward, the drive shaft 66 rotates in an opposite direction but the second one-way bearing 74 prevents corresponding rotation of the second transfer gear 70. To this end, the trigger 26 can return to the position shown in FIG. 5C without causing advancement of the plunger 24. The process may then be repeated until the plunger 24 is fully retracted from the tube 12. As described above, the lumen 22 may then be recharged with another dose of fluid via the Y-shaped receptacle 16 if the fluid dispensing device 10 is to be reused.

Advantageously, the plunger 24 is configured to be flexible so that the plunger 24 may be moved from a straightened configuration within the lumen 22 to a curved or coiled configuration within the housing 14. To this end, the plunger 24 is flexible enough to be coiled about the reel 40 in this embodiment of the fluid dispensing device 10. The plunger 24 therefore may be composed of an elastomeric head 24c in the distal portion 24b configured to press on fluid and a braided stainless steel cord or wire extending rearward of the elastomeric head 24c. In one example, the steel cord may have an outer diameter of about 2 to 3 millimeters such that the gripper wheel 44 can reliably grip the plunger 24 while permitting full coiling on the reel 40. Alternatively, the plunger 24 may be composed of a plastic material or other readily bendable materials in other embodiments. The flexibility of the plunger 24 permits the total length of the fluid dispensing device 10 to be much shorter than conventional fluid dispensing devices. Consequently, the fluid dispensing device 10 is easier to manipulate and use in a surgery setting.

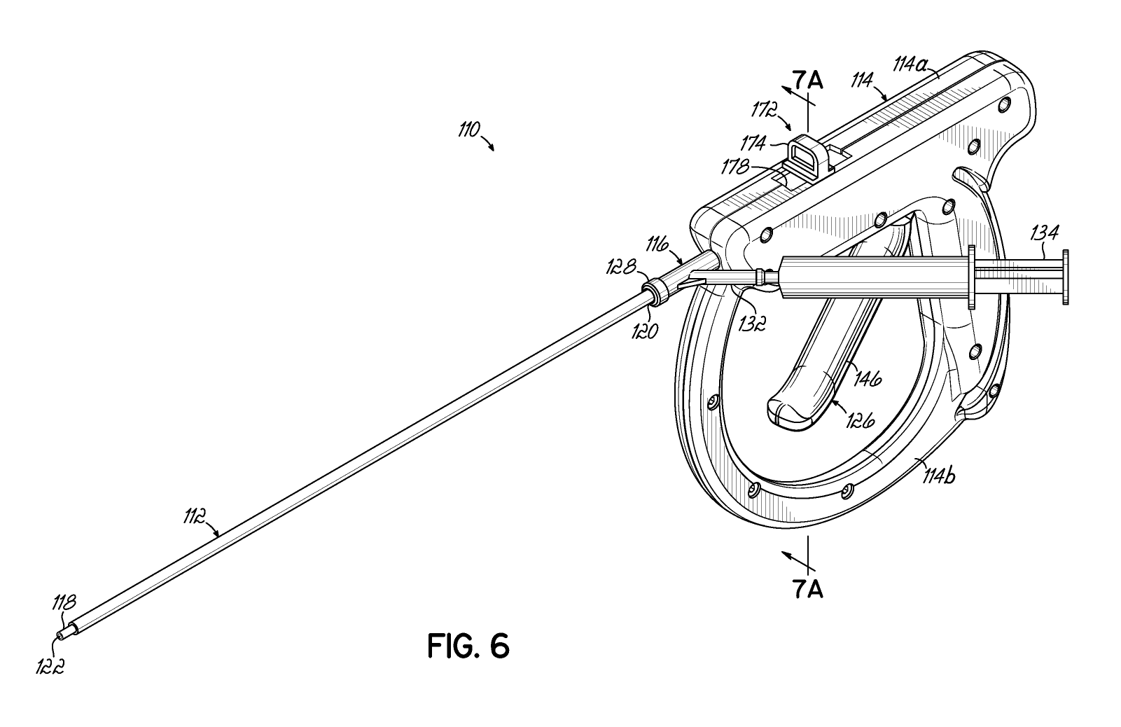

FIGS. 6-12B illustrate another embodiment of a fluid dispensing device 110 configured to deliver a dose of fluid into a surgical site. Similar to the previous embodiment, the fluid dispensing device 110 of this embodiment is operable to dispense the dose of fluid into small incision surgical sites such as those used in minimally-invasive surgery and laparoscopic procedures. As shown in FIG. 6, the fluid dispensing device 110 includes an elongate tube 112 for holding the dose of fluid, a housing 114, and a Y-shaped receptacle 116 coupling the elongate tube 112 to the housing 114. The elongate tube 112 is generally rigid and includes a distal end 118, a proximal end 120 coupled to the Y-shaped receptacle 116, and a lumen 122 extending from the distal end to the proximal end 120. The housing 114 is configured to receive a plunger 124 (not shown in FIG. 6) that may be advanced into the lumen 122 to force the dose of fluid from the distal end 118 of the tube 112. The plunger 124 may also be retracted from the lumen 122 to permit a new dose of fluid to be delivered into the lumen 122. The fluid dispensing device 110 also includes a trigger 126 coupled to the housing 114 and operable to advance or retract the plunger 124 as described in further detail below.

Also shown in FIG. 6, the Y-shaped receptacle 116 includes a first end 128 configured to receive the proximal end 120 of the tube 112, a second end 130 configured to be received in the housing 114, and an auxiliary port 132 between the first and second ends 128, 130. The auxiliary port 132 is in fluid communication with the lumen 122 when the tube 112 is coupled to the Y-shaped receptacle 116. As such, the auxiliary port 132 may be coupled to a syringe 134 or other known medicament container to reload the elongate tube 112 with fluid when the plunger 124 is retracted. It will be understood that the elongate tube 112 may be directly coupled to the housing 114 without the Y-shaped receptacle 116 in other non-illustrated embodiments.

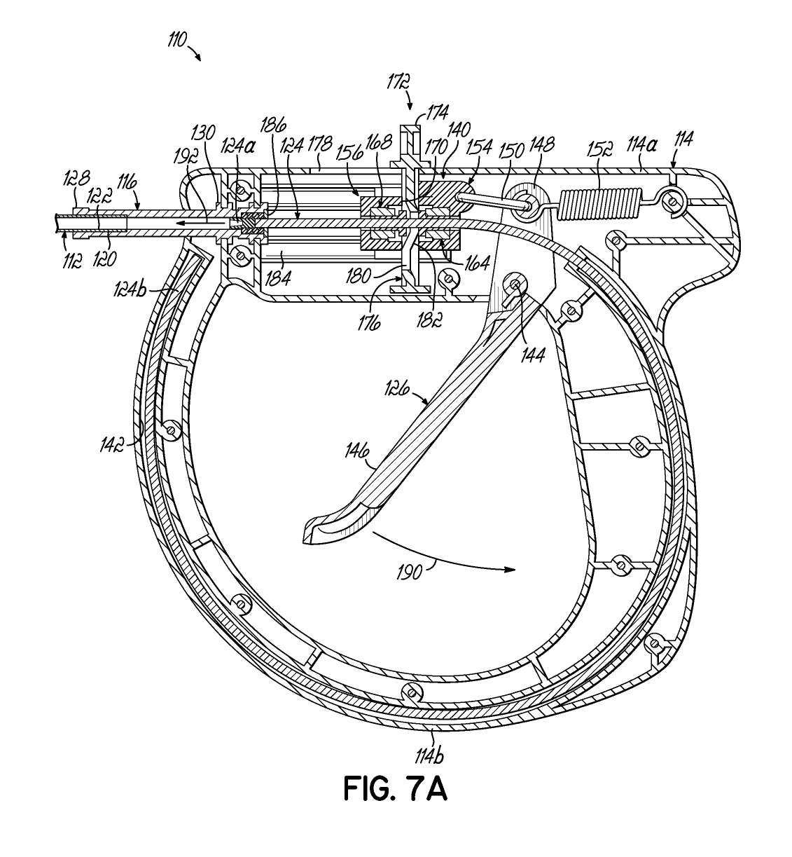

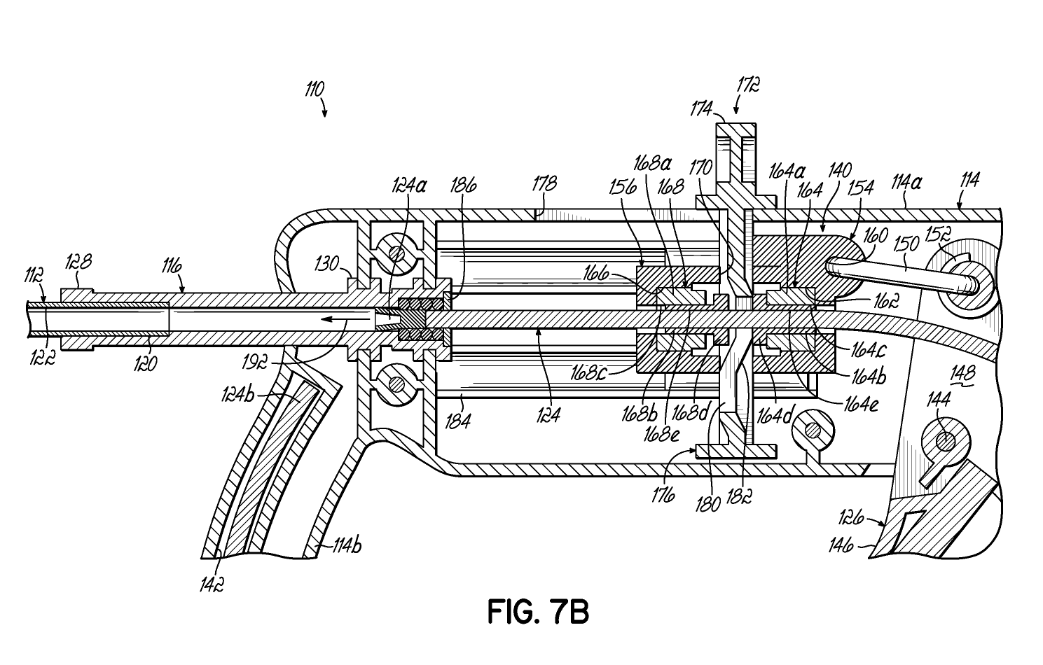

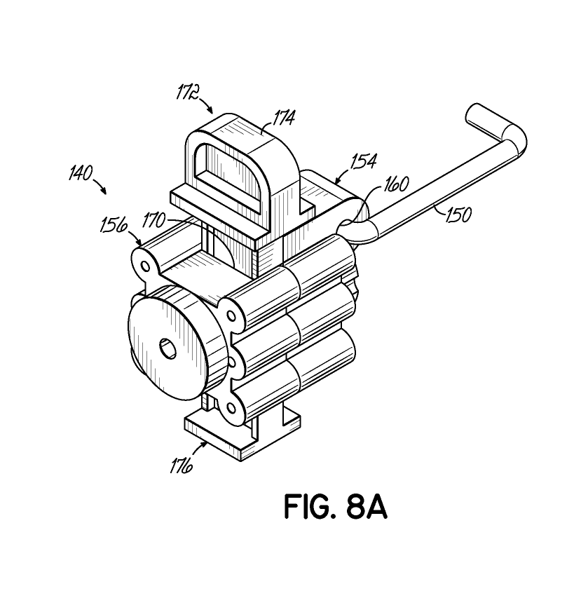

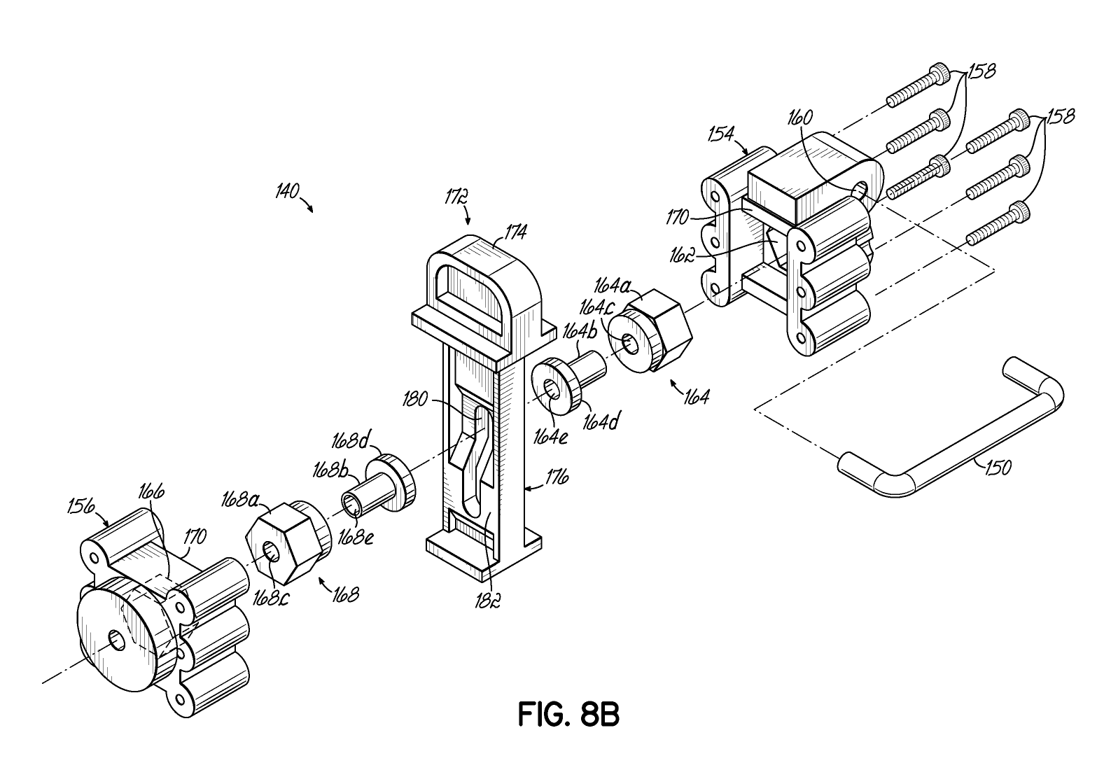

The interior drive components of the fluid dispensing device 110 are shown more clearly in FIGS. 7A-8B. As shown in FIG. 7A, the plunger 124 includes a distal portion 124a configured to press on fluid in the lumen 122 and a proximal portion 124b at least partially disposed in the housing 114 and configured to be moved from a straightened configuration in the lumen 122 to a curved or coiled configuration in the housing 114. To accommodate the plunger 124 in the curved configuration, the housing 114 is shaped as a loop with the trigger 126 extending into the center of the loop. More specifically, the housing 114 includes an upper portion 114a configured to contain an actuation device 140 and a lower portion 114b shaped as a loop and configured as a handle. The lower portion 114b of the housing 114 may further include a storage channel 142 configured to receive the plunger 124 when retracted within the housing 114 as shown in FIG. 7A. Although the storage channel 142 does not extend through the upper portion 114a of the housing 114 in the illustrated embodiment, it will be understood that the storage channel 142 could be extended in other embodiments to accommodate more than one coil of the plunger 124.

The trigger 126 is pivotally coupled to the housing 114 at a pivot point 144. The trigger 126 includes a handle portion 146 extending downwardly from the pivot point 144 and an internal portion 148 extending upwardly from the pivot point 144. It will be appreciated that the use of positional or directional terms such as upper, lower, internal, and external are used for illustrative purposes only in this description and do not limit the scope of this invention. The internal portion 148 of the trigger 126 is coupled to a C-shaped connector member 150 and a biasing member such as a tension spring 152. The tension spring 152 is also coupled to the housing 114 and pulls the internal portion 148 of the trigger 126 toward the position shown in FIG. 7A. The connector member 150 couples the internal portion 148 of the trigger 126 to the actuation device 140, which moves the plunger 124. Thus, the connector member 150 transfers movement force from the trigger 126 through the actuation device 140 to the plunger 124 as described in greater detail below.

The actuation device 140 is more clearly shown in FIGS. 7B, 8A, and 8B. The actuation device 140 includes a first body portion 154 and a second body portion 156 configured to be coupled by a plurality of fasteners 158 such as threaded fasteners 158 shown in FIG. 8B. The first body portion 154 includes a connector aperture 160 configured to receive a portion of the connector member 150. The first body portion 154 also includes an internal aperture 162 facing toward the second body portion 156 and shaped to receive a first gripping collar 164. Similarly, the second body portion 156 also includes an internal aperture 166 facing toward the first body portion 154 and shaped to receive a second gripping collar 168. In this regard, the first and second gripping collars 164, 168 are arranged in opposing directions in the actuation device 140. The first and second body portions 154, 156 also collectively define a switch channel 170 when assembled for receiving a switch member 172.

Each of the gripping collars 164, 168 may include a known cable quick connect collar with gripping teeth or similar structure that enables the plunger 124 to freely slide only in one direction when the gripping collar 164, 168 is engaged. Alternatively, and as shown in the exemplary embodiment shown in FIGS. 6-12B, the first gripping collar 164 includes a collar receptacle 164a and a collar member 164b. The collar receptacle 164a is shaped to be received in the internal aperture 162 of the first body portion 154 and includes a receptacle aperture 164c for receiving the collar member 164b. The collar member 164b includes a head portion 164d facing the switch member 172 and a gripping aperture 164e. The gripping aperture 164e is nominally sized to permit free sliding movement of the plunger 124 in either direction through the collar member 164b. The receptacle aperture 164c is slightly tapered such that when the collar member 164b is fully inserted into the collar receptacle 164a, the collar receptacle 164a effectively clamps the collar member 164b inwardly onto the plunger 124. When the collar member 164b is so clamped on the plunger 124, the gripping aperture 164e is adapted to permit free sliding movement of the plunger 124 through the first gripping collar 164 in only one direction (to the left as shown in FIG. 7B). To this end, when the first gripping collar 164 is engaged, the plunger 124 may only be driven to advance into the lumen 122 by movement of the actuation device 140.

Similarly, the second gripping collar 168 includes a collar receptacle 168a and a collar member 168b. The collar receptacle 168a is shaped to be received in the internal aperture 166 of the second body portion 156 and includes a receptacle aperture 168c for receiving the collar member 168b. The collar member 168b includes a head portion 168d facing the switch member 172 and a gripping aperture 168e. The gripping aperture 168e is nominally sized to permit free sliding movement of the plunger 124 in either direction through the collar member 168b. The receptacle aperture 168c is slightly tapered such that when the collar member 168b is fully inserted into the collar receptacle 168a, the collar receptacle 168a effectively clamps the collar member 168b inwardly onto the plunger 124. When the collar member 168b is so clamped on the plunger 124, the gripping aperture 168e is adapted to permit free sliding movement of the plunger 124 through the second gripping collar 168 in only one direction (to the right as shown in FIG. 7B). To this end, when the second gripping collar 168 is engaged, the plunger 124 may only be driven to retract from the lumen 122 by movement of the actuation device 140.

The switch member 172 includes a switch handle 174 configured to be disposed outside of the housing 114 and an actuation portion 176 extending below the switch handle 174 and through the switch channel 170 in the actuation device 140. The housing 114 includes a top aperture 178 configured to receive the switch handle 174 and permit forward and rearward sliding of the switch member 172 with the actuation device 140. The actuation portion 176 includes a central aperture 180 configured to receive the plunger 124 and an angled surface 182 adjacent the central aperture 180. The angled surface 182 is configured to actively push one of the collar members 164b, 168b of the first or second gripping collars 164, 168 into the corresponding collar receptacle 164a, 168a depending on the vertical position of the actuation portion 176 within the switch channel 170. The vertical position of the actuation portion 176 may be modified by manually applying force upwardly or downwardly on the switch handle 174. The specific operation of the switch member 172 and the first and second gripping collars 164, 168 is described in further detail below.

In addition to the top aperture 178, the upper portion 114a of the housing 114 includes a bearing channel 184 configured to receive the actuation device 140 and allow free sliding of the actuation device 140 along the upper portion 114a of the housing 114. The bearing channel 184 may communicate with a plunger outlet 186 configured to permit passage of the plunger 124 out of the housing 114. Furthermore, the plunger outlet 186 is configured to seal the exterior of the housing 114 from the interior. As described with reference to the previous embodiment, the plunger 124 is designed to be flexible and may be composed of braided stainless steel wire, plastic, or another material configured to bend. Thus, the plunger 124 may be advanced into the lumen 122 to force the dose of fluid out of the tube 112, and then retracted into the housing 114 in a curved or coiled state. The distal portion 124a of the plunger 124 may also be formed of an elastomeric material to more readily force fluid through the lumen 122 as in the previous embodiment.

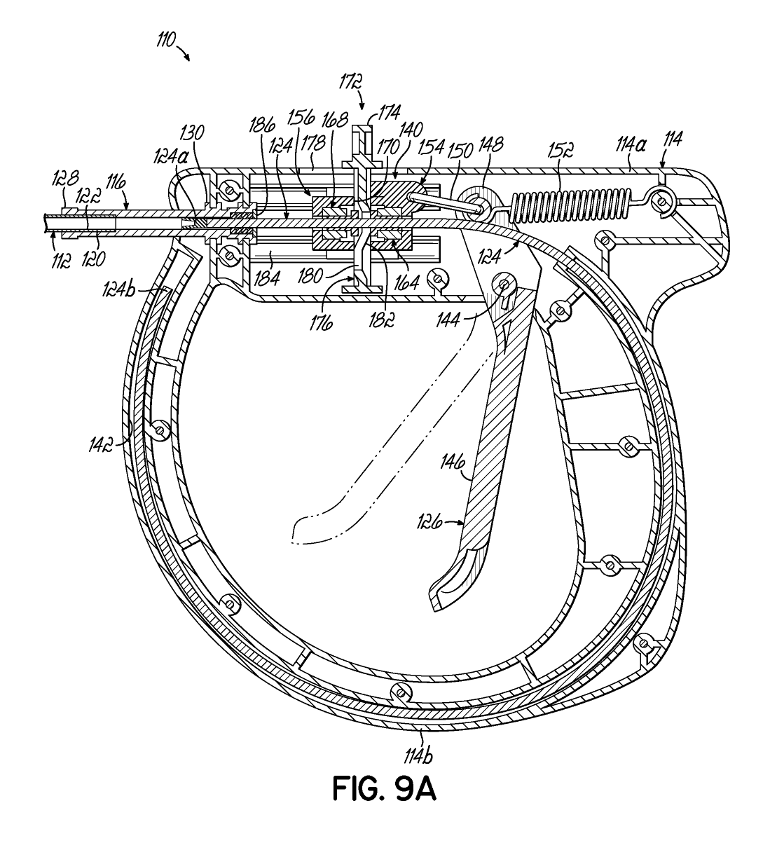

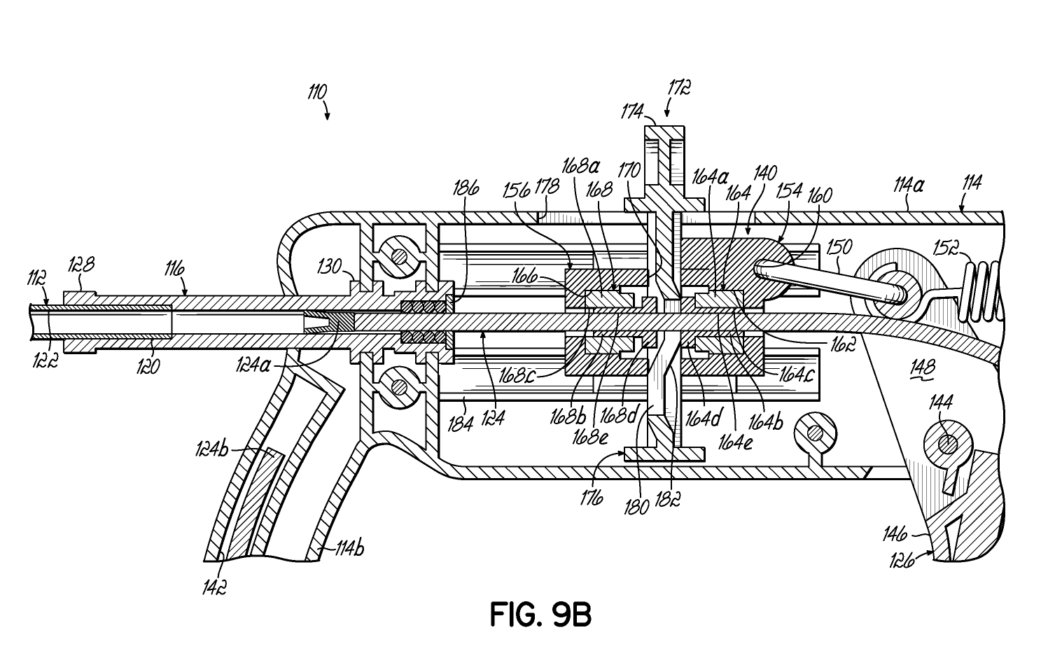

The actuation device 140 is illustrated in a first operational mode in FIGS. 7A-9B. In this first operational mode, the switch member 172 is disposed in a lower position in which the angled surface 182 actively forces the collar member 164b of the first gripping collar 164 into the collar receptacle 164a such that the first gripping collar 164 actively engages the plunger 124. The trigger 126 and the actuation device 140 begin in the position shown in FIG. 7A. The handle portion 146 of the trigger 126 is then pulled back as shown by arrow 190 such that the actuation device 140 is driven forward. With the first gripping collar 164 actively engaging the plunger 124, this forward movement of the actuation device 140 is also transferred to advance the plunger 124 as indicated by arrow 192. After the trigger 126 is pulled back to the position shown in FIGS. 9A and 9B, the plunger 124 has advanced toward the lumen 122 as shown. When the tension spring 152 forces the trigger 126 back to the original position, the actuation device 140 and first gripping collar 164 will freely slide along the plunger 124 such that the plunger 124 is not immediately retracted. Thus, in the first operational mode, movement of the trigger 126 only advances the plunger 124.

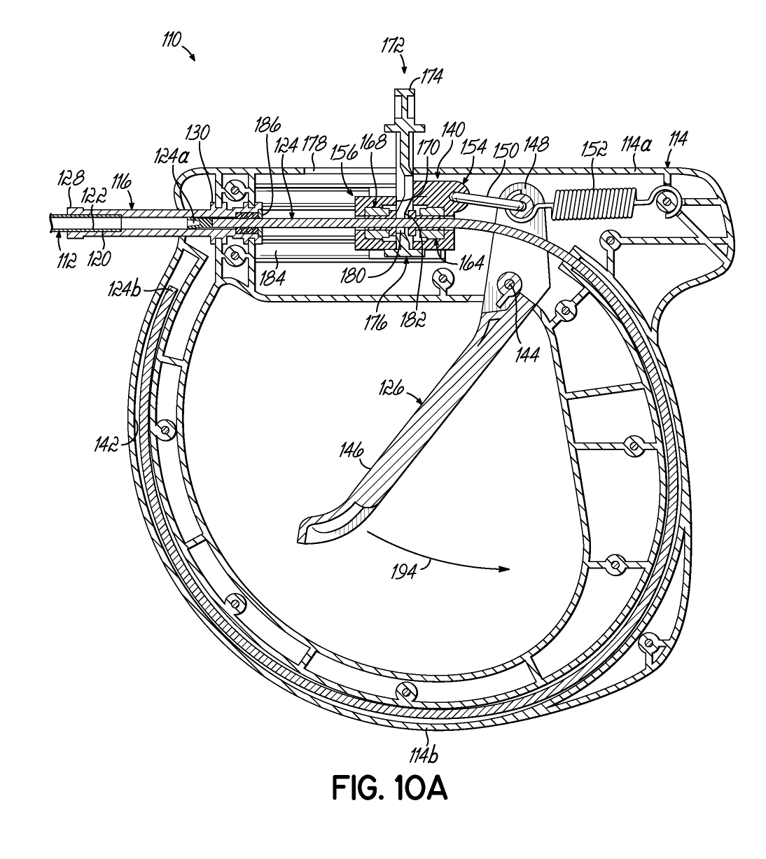

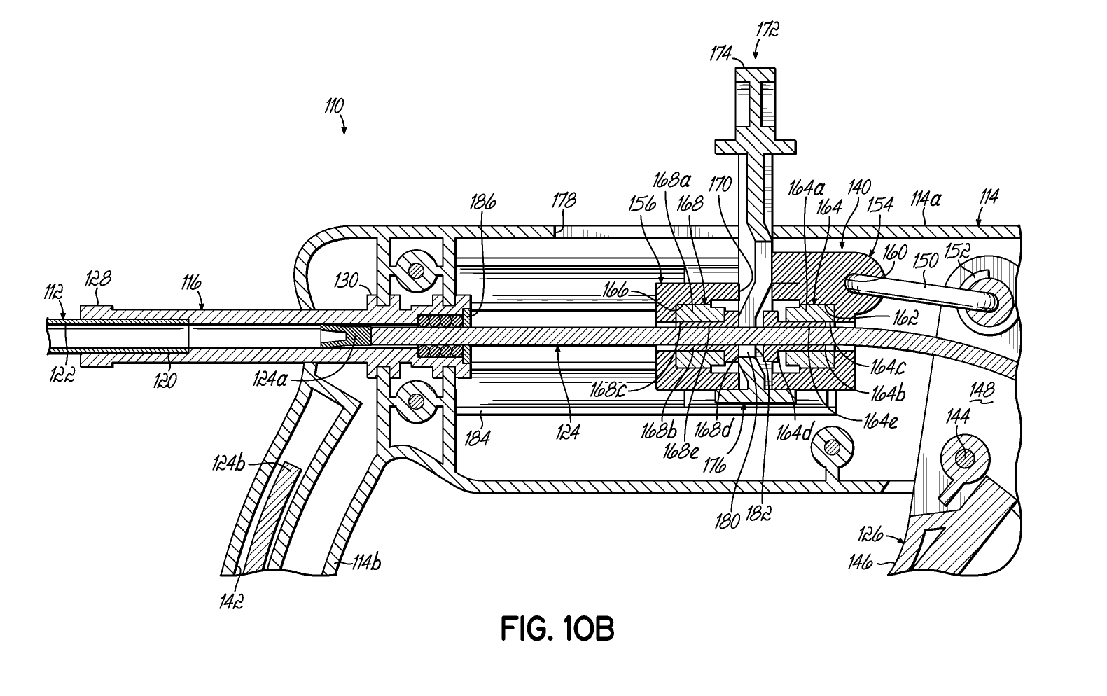

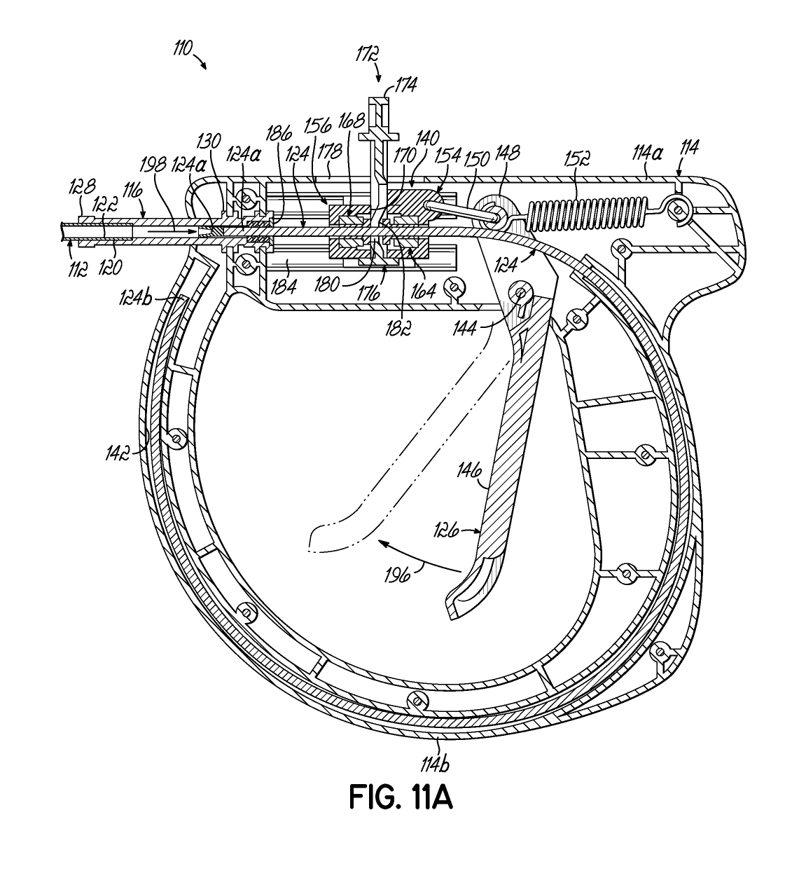

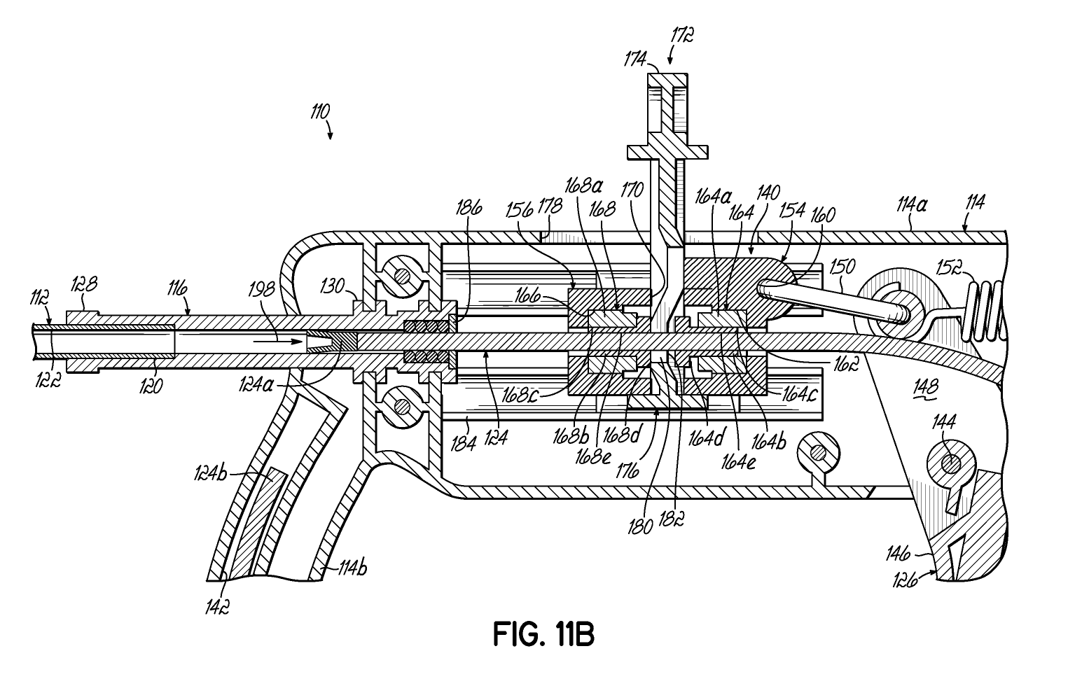

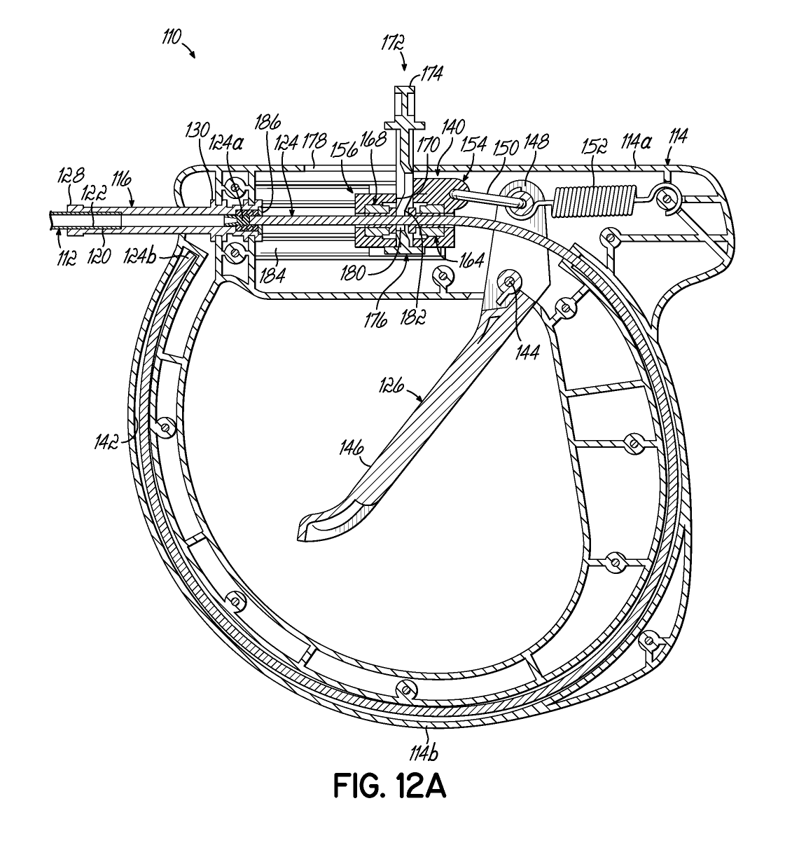

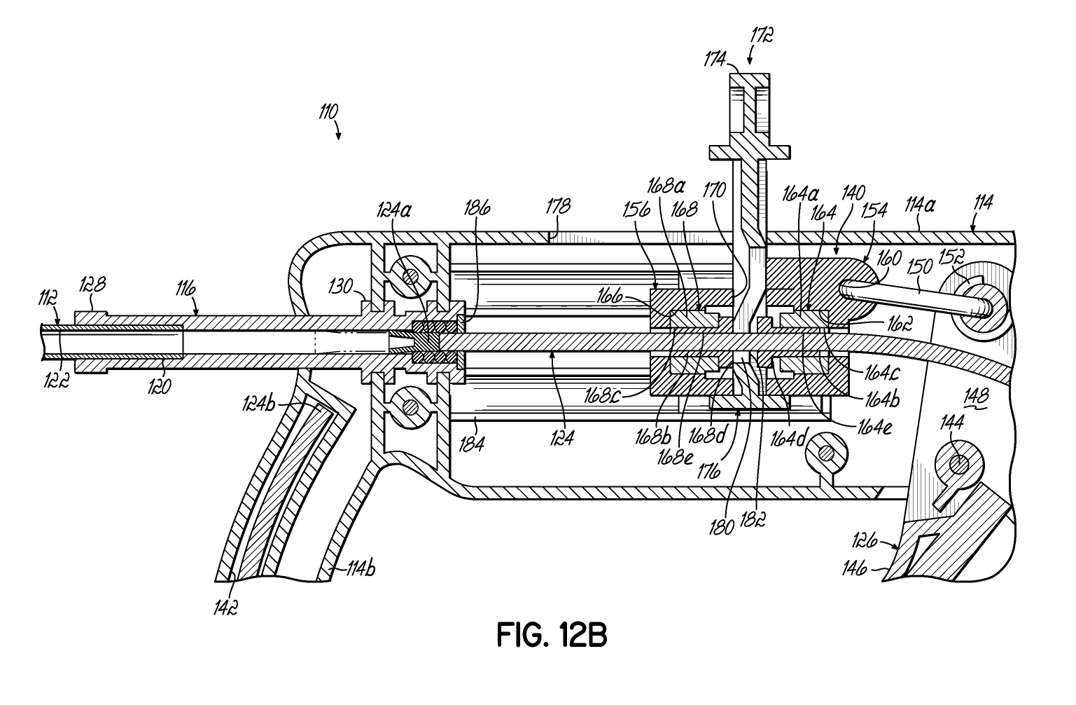

The actuation device 140 is illustrated in a second operational mode in FIGS. 10A-12B. In the second operational mode, the switch member 172 is disposed in an upper position in which the angled surface 182 actively forces the collar member 168b of the second gripping collar 168 into the collar receptacle 168a such that the second gripping collar 168 actively engages the plunger 124. The trigger 126 and the actuation device 140 begin in the position shown in FIG. 10A. The handle portion 146 of the trigger 126 is then pulled back as shown by arrow 194 such that the actuation device 140 is driven forward. When the second gripping collar 168 is actively engaged with the plunger 124, the actuation device freely slides along the plunger 124 when traveling in a forward direction. As such, when the trigger is pulled back to the position shown in FIG. 11A, the actuation device 140 has slid forward but the plunger 124 has not moved. The tension spring 152 then forces the trigger 126 back to the original position as indicated by arrow 196. With the second gripping collar 168 actively engaging the plunger 124, the resulting rearward movement of the actuation device 140 is also transferred to retract the plunger 124 as indicated by arrow 198. After the trigger 126 is forced back to the position shown in FIGS. 12A and 12B, the plunger 124 has retracted away from the lumen 122 as shown. Therefore, in the second operational mode, movement of the trigger 126 only retracts the plunger 124.

Once again, the flexibility of the plunger 124 permits the total length of the fluid dispensing device 110 to be much shorter than conventional fluid dispensing devices. Furthermore, the fluid dispensing device 110 is readily and easily switched between a first operational mode for advancing the plunger 124 and a second operational mode for retracting the plunger 124. As such, the fluid dispensing device 110 of this embodiment is also easier to manipulate and use in a surgery setting.

While the present invention has been illustrated by the description of specific embodiments thereof, and while the embodiments have been described in considerable detail, it is not intended to restrict or in any way limit the scope of the appended claims to such detail. The various features discussed herein may be used alone or in any combination. Additional advantages and modifications will readily appear to those skilled in the art. The invention in its broader aspects is therefore not limited to the specific details, representative apparatus and methods and illustrative examples shown and described. Accordingly, departures may be made from such details without departing from the scope or spirit of the general inventive concept.

What is claimed is:

1. A fluid dispensing device configured to deliver a dose of fluid into a surgical site, the device comprising:
a tube including a proximal end, a distal end, and a lumen extending from the proximal end to the distal end, the lumen configured to receive the dose of fluid;
a housing coupled to the tube at the proximal end;
a flexible plunger at least partially disposed in the housing, the flexible plunger including a proximal portion and a distal portion, the proximal portion of the flexible plunger having a curved configuration when disposed within the housing;
an actuation device coupled with the housing and engaging the flexible plunger; and
a trigger coupled for movement relative to the housing, the trigger operable to cause the actuation device to move the distal portion of the flexible plunger into an extended configuration within the lumen to force the dose of fluid out of the distal end of the tube.

2. The fluid dispensing device of claim 1, wherein the actuation device includes a first operational mode in which movement of the trigger causes advancement of the flexible plunger into the lumen and a second operational mode in which movement of the trigger causes retraction of the flexible plunger from the lumen.

3. The fluid dispensing device of claim 2, wherein the actuation device includes first and second actuating members operatively coupled to the trigger, the first actuating member configured to transfer movement of the trigger to advance the flexible plunger in the first operational mode, the second actuating member configured to transfer movement of the trigger to retract the flexible plunger in the second operational mode.

4. The fluid dispensing device of claim 3, wherein the actuation device includes a drive shaft and at least two transfer gears, the first actuating member includes a first one-way bearing selectively coupling one of the transfer gears to the drive shaft in the first operational mode, and the second actuating member includes a second one-way bearing selectively coupling another of the transfer gears to the drive shaft in the second operational mode.

5. The fluid dispensing device of claim 3, wherein the first actuating member includes a first gripping collar configured to engage the flexible plunger in the first operational mode, and the second actuating member includes a second gripping collar configured to engage the flexible plunger in the second operational mode.

6. (canceled)

7. The fluid dispensing device of claim 1, wherein the proximal portion of the flexible plunger is coiled within the housing.

8. The fluid dispensing device of claim 1, further comprising:
a Y-shaped receptacle coupling the proximal end of the tube to the housing, the receptacle including an auxiliary inlet port configured to direct a new dose of fluid into the lumen when the flexible plunger is retracted from the lumen.

9. A fluid dispensing device configured to deliver a dose of fluid into a surgical site, the device comprising:
a tube including a proximal end, a distal end, and a lumen extending from the proximal end to the distal end, the lumen configured to receive the dose of fluid;
a housing coupled to the tube at the proximal end;
a plunger at least partially disposed in the housing, the plunger including a proximal portion and a distal portion;
an actuation device coupled with the housing and engaging the plunger; and
a trigger coupled for movement relative to the housing, the trigger operable to cause the actuation device to move the distal portion of the plunger,
wherein the actuation device includes a first operational mode in which movement of the trigger causes advancement of the plunger into the lumen and a second operational mode in which movement of the trigger causes retraction of the plunger from the lumen,
wherein the actuation device includes first and second actuating members operatively coupled to the trigger, the first actuating member configured to transfer movement of the trigger to advance the plunger in the first operational mode, the second actuating member configured to transfer movement of the trigger to retract the plunger in the second operational mode.

10. (canceled)

11. The fluid dispensing device of claim 9, wherein the actuation device includes a drive shaft and at least two transfer gears, the first actuating member includes a first one-way bearing selectively coupling one of the transfer gears to the drive shaft in the first operational mode, and the second actuating member includes a second one-way bearing selectively coupling another of the transfer gears to the drive shaft in the second operational mode.

12. The fluid dispensing device of claim 9, wherein the first actuating member includes a first gripping collar configured to engage the plunger in the first operational mode, and the second actuating member includes a second gripping collar configured to engage the plunger in the second operational mode.

13. The fluid dispensing device of claim 9, wherein the proximal portion of the plunger is curved when disposed in the housing.

14. The fluid dispensing device of claim 9, wherein the proximal portion of the plunger is coiled within the housing.

15. The fluid dispensing device of claim 9, further comprising:
a Y-shaped receptacle coupling the proximal end of the tube to the housing, the receptacle including an auxiliary inlet port configured to direct a new dose of fluid into the lumen when the plunger is retracted from the lumen.

16. A method of dispensing a dose of fluid into a surgical site with a fluid dispensing device including a tube having a lumen holding the dose of fluid, a housing coupled to the tube, a flexible plunger including a distal portion and a proximal portion which is curved when disposed in the housing, an actuation device engaging the flexible plunger, and a trigger operatively coupled to the actuation device, the method comprising:
placing the actuation device in a first operational mode in which movement of the trigger causes advancement of the flexible plunger into the lumen;
actuating the trigger to move the actuation device; and
moving the proximal portion of the flexible plunger with the actuation device from a curved configuration within the housing to an extended configuration within the lumen to force the dose of fluid from the lumen.

17. The method of claim 16, further comprising:
placing the actuation device in a second operational mode in which movement of the trigger causes retraction of the flexible plunger from the lumen;
actuating the trigger to move the actuation device; and
moving the proximal portion of the flexible plunger with the actuation device from an extended configuration within the lumen to a curved configuration within the housing.

18. The method of claim 17, wherein the fluid dispensing device includes a Y-shaped receptacle coupling the tube to the housing, the receptacle including an auxiliary inlet port, and the method further comprises:
directing a dose of fluid into the lumen when the flexible plunger is retracted from the lumen.

19. The fluid dispensing device of claim 1, wherein the housing further comprises a reel configured to receive the proximal portion of the flexible plunger when the proximal portion is in the curved configuration within the housing.

20. The fluid dispensing device of claim 19, wherein the reel is positioned proximally of the actuation device within the housing.

21. The fluid dispensing device of claim 1, wherein the housing further comprises an upper portion containing the actuation device and a lower portion configured as a loop-shaped handle surrounding the trigger.

22. The fluid dispensing device of claim 21, wherein the lower portion of the housing further includes an arcuate storage channel configured to receive the flexible plunger when the plunger is in the curved configuration within the housing.
